(19) United States
(12) Patent Application Publication
(10) Pub. No.: US 2019/0343827 A1
(43) Pub. Date: Nov. 14, 2019

(54) DEUTERATED ANALOGS OF TARIQUIDAR

(71) Applicant: Izumi Technology, LLC, Lexington, MA (US)

(72) Inventor: Antonius Martinus Gustave Bunt, Lexington, MA (US)

(73) Assignee: Izumi Technology, LLC, Lexignton, MA (US)

(21) Appl. No.: 16/406,078

(22) Filed: May 8, 2019

Related U.S. Application Data
(60) Provisional application No. 62/668,780, filed on May 8, 2018.

(51) Int. Cl. A61K 31/4725; A61K 31/277; C07D 401/12

(52) U.S. Cl. CPC A61K 31/4725; C07D 401/12; A61K 31/277

(57) ABSTRACT

The present invention relates to efflux inhibitor compounds, compositions, and methods of using the same. More specifically, the instant invention comprises deuterated analogs of tariquidar with superior pharmacokinetic properties such that it is now possible to facilitate accumulation and distribution of therapeutic agents to effective levels in cells or compartments protected by efflux transporter proteins such as P-Glycoprotein (P-GP) and Breast Cancer Resistance Protein (BCRP). Such pump protected compartments include brain, spinal cord, nerves, cerebrospinal fluid, testis, eyeballs, retina, inner ear, placenta, mammary gland, liver, biliary tract, kidney, intestines, lung, adrenal cortex, endometrium, hematopoietic cells, stem cells, and solid tumors. In other embodiments, the present invention comprises methods of using the instant deuterated analogs.

### TECHNICAL FIELD

The present invention relates to deuterated active pharmaceutical ingredients and to efflux inhibitors.

### BACKGROUND

Tariquidar, sometimes referred to as XR9576, is a compound with the structure of N-[2-[[4-[2-(6,7-dimethoxy-3,4-dihydro-1H-isoquinolin-2-yl)ethyl]phenyl]carbamoyl]-4,5-dimethoxyphenyl]quinoline-3-carboxamide 003 Tariquidar is recognized as a dual P-gp/BCRP inhibitor, as reported by Mistry P, Stewart A J, Dangerfield W, Okiji S, Liddle C, Bootle D, Plumb J A, Templeton D, Charlton P: In vitro and in vivo reversal of P-glycoprotein-mediated multidrug resistance by a novel potent modulator, XR9576. Cancer Res 2001, 61:749-758. See also Kannan et al., ACS Chem Neurosci. 2011 Feb. 16; 2(2):82-9.

It is well-recognized that certain cells and compartments in the body contain pumps such as P-gp and BCRP that pump substrates out of such compartments. In some cases, certain drugs can be substrates and such pumps prevent accumulation of such drugs to therapeutic levels in those compartments. Based on tariquidar's inhibitory effect on P-gp and BCRP, it was speculated that co-administering tariquidar with drugs that are pump substrates may allow accumulation of such drugs to therapeutic levels in pump-protected compartments and enhance the efficacy response. For reasons not well understood, such co-therapeutic attempts to date have been disappointing.

Tariquidar was co-administered with doxorubicin, vinorelbine, or docetaxel in children and adolescents with refractory solid tumors. While the clearance of docetaxel and vinorelbine was reduced compared to historic data (in absence of tariquidar), the clinical response was low. Out of 29 patients, only one had a complete and two had a partial response (Fox et al. Cancer Chemother Pharmacol December 2015, Volume 76, Issue 6, pp 1273-1283)

In another study, co-administration of tariquidar and paclitaxel or carboplatin in non-small-cell lung cancer was terminated early due to toxicity (Fox et al., Expert Rev Anticancer Ther 2007 April; 7(4):447-59).

In another study, tariquidar was co-administered with docetaxel in patients with lung, ovarian and cervical cancer. No significant differences in docetaxel disposition was observed as a result of tariquidar co-administration. (Kelly et al, Clin Cancer Res. 2011 Feb. 1; 17(3): 569-580.)

Because of the great unpredictability in the art and poor correlations in many cases between animal and human data, it is difficult to predict a path to improving tariquidar efficacy to enhance accumulation of drugs in compartments that contain the P-gp and BCRP pumps. Among the many approaches often pursued are variations in dose administration, route of administration, formulations, compound complexation, and compound modification.

A potentially attractive strategy for improving metabolic stability of some drugs is deuterium modification. In this approach, one attempts to slow the CYP-mediated metabolism of a drug or to reduce the rate of formation of inactive metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the absorption, distribution, metabolism, excretion and/or toxicity (‘ADMET’) properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds, deuteration indeed caused decreased metabolic clearance in vivo. For others, no change in metabolism was observed. Still others demonstrated increased metabolic clearance. The great unpredictability and variability in deuterium effects has led experts to question or dismiss deuterium modification as a viable drug design strategy for inhibiting metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

What is needed in the art are new methods, formulations, compounds, or compound analogs that can effectively enhance accumulation of drugs in compartments that are protected by efflux pumps such as PgP and BCRP.

### SUMMARY OF THE INVENTION

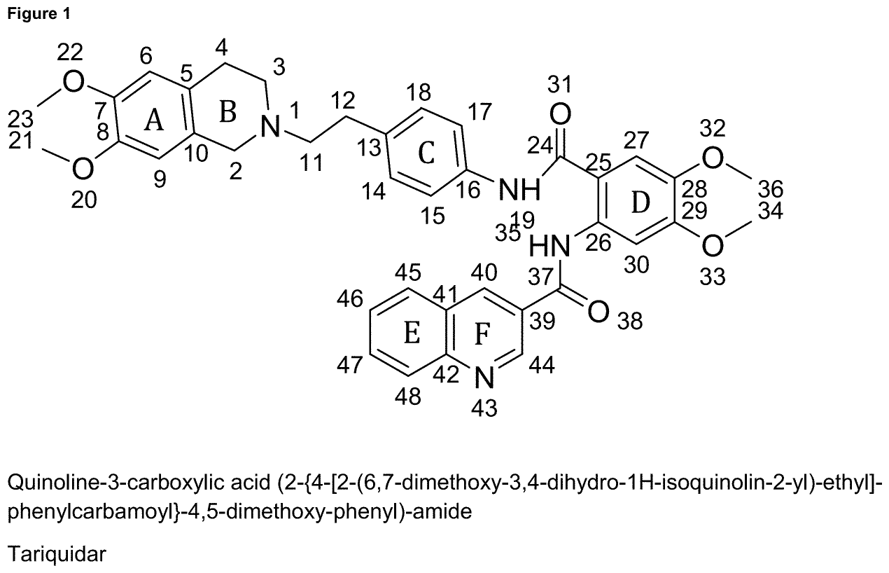

This present invention provides improved compounds that are deuterated analogs of tariquidar. In one embodiment, a deuterated analog of the invention comprises tariquidar comprising one or two or three or four or five, or six, or seven or eight or nine or ten or 11 or 12 or 13 or 14 or 15 or 16 or 17 or 18 or 19 or 20 or more deuterium atoms, wherein, collectively, the substitutions are located at one or more of locants 1-48 as designated in FIG. 1. In other embodiments, instant analogs comprise 1-2 deuterium substitutions at locant 4 and optionally one or more deuterium substitutions at locants 1-48. The instant invention also includes compositions comprising the instant analogs and methods of using the same.

The various instant deuterated analogs of tariquidar are represented by formula 1:

or a pharmaceutically acceptable salt thereof, comprising at least one deuterium atom wherein:

each Y is independently selected from hydrogen or deuterium; and

each R is independently selected from CH₃, CH₂D₁, CH₁D₂, and CD₃.

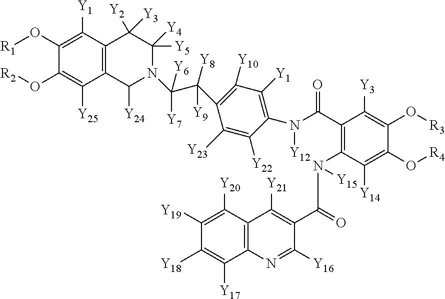

### BRIEF DESCRIPTION OF THE FIGS

FIG. 1 shows tariquidar with locant numbers.

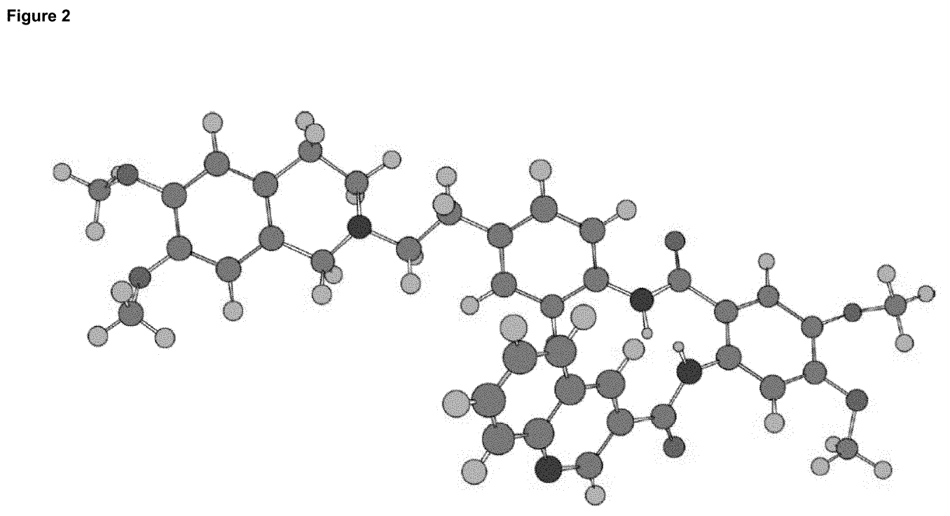

FIG. 2 shows a 3D rendition of Tariquidar and each hydrogen which can be substituted with deuterium according to the present invention.

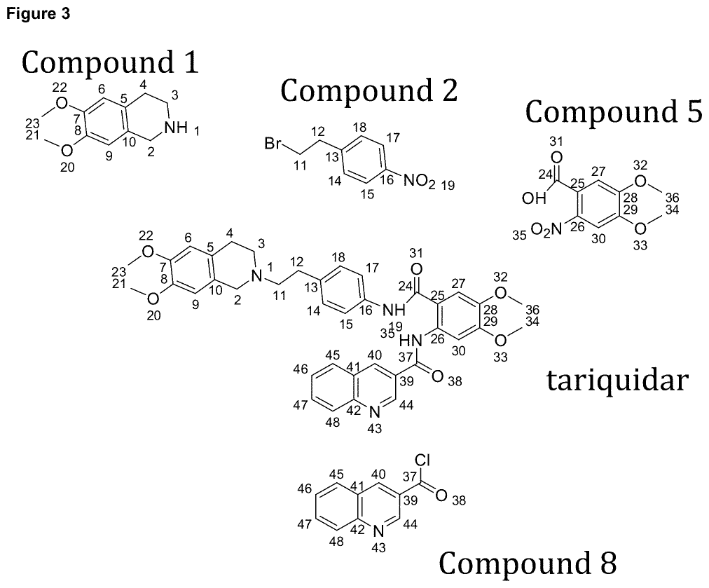

FIG. 3 shows reactant compounds for synthesis of tariquidar and tariquidar analogs. Locant numbers reference the final tariquidar product.

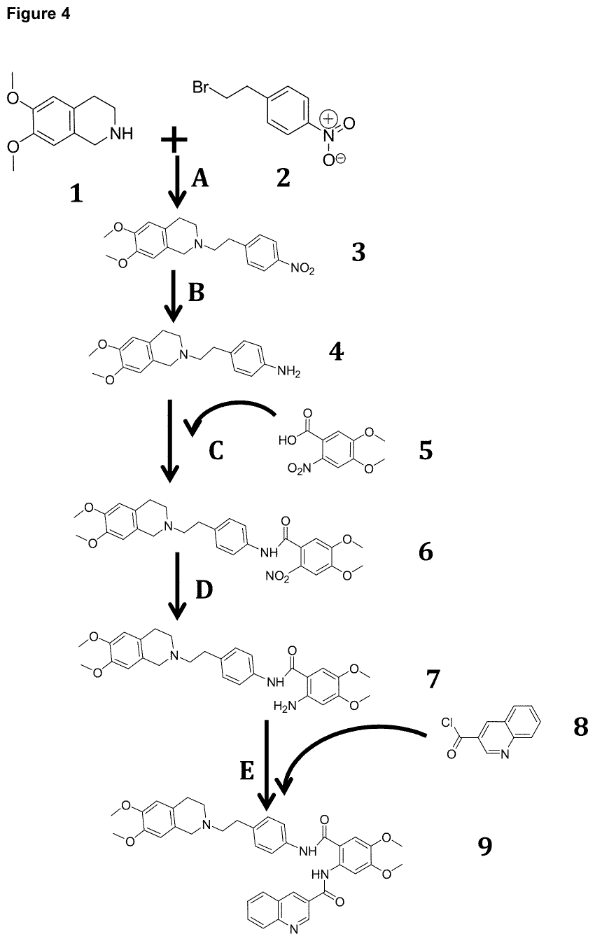

FIG. 4 shows a representative method of tariquidar synthesis.

### DETAILED DESCRIPTION OF THE INVENTION

As used here, the following definitions and abbreviations apply.

“AUC” or “AUC 0-00” is the area under the curve from time 0 extrapolated to infinite time.

“Co-administered” (or “co-therapy” or “in combination with”) in reference to instant analogs or compositions with therapeutic agent(s), is meant to include the administration of such analogs or composition in a single dosage form, in separate dosage forms at the same time, or in separate dosage forms and different times. Optionally, co-administration can be provided in any manner that results in a second therapeutic agent being present in a subject at the same time as the tariquidar analog.

“DMF” means N,N-Dimethylformamide

“Deuterium atoms substituted” or “deuterium atoms are substituted”, as used herein, refers to a deuterium atom that is substituted for a hydrogen atoms.

“Tariquidar” means N-[2-[[4-[2-(6,7-dimethoxy-3,4-dihydro-1H-isoquinolin-2-yl)ethyl]phenyl]carbamoyl]-4,5-dimethoxyphenyl]quinoline-3-carboxamide. The term “tariquidar” is also meant to embrace the compound of the same structure described above, excepting for one or more hydrogens being replaced by a deuterium; such a compound is also referred to as a deuterated analog of tariquidar or a tariquidar analog. Unless otherwise clear by the context, the term “tariquidar” means an unsubstituted tariquidar.

“Tariquidar analog(s)” or “instant analog” means a tariquidar analog of the instant invention.

“Isotopologue”, as used herein, refers to a species in which the chemical structure differs from a specific compound of this invention only in the isotopic composition thereof.

“Isotopic enrichment factor”, as used herein, means the ratio of the isotopic abundance in an instant analog and the natural abundance of a specified isotope (e.g. deuterium).

“Isotopic purity”, as used herein, is the percentage of analogs molecules in a composition that contain the designated number of deuterated atoms at the designated deuteration sites.

“Kp, brain”, as used here, is the brain-to-plasma partition coefficient as measured, e.g. as set forth in Sane et al. Drug Metabolism And Disposition vol. 40 no. 8 1612-1619 for elacridar.

“Normal valence”, when used in reference to the instant analogs, refers to the combining power of an element as measured by the number of hydrogen plus deuterium atoms it can displace or combine with. For the sake of clarity, the normal valence of carbon is 4, of oxygen is 2, and of nitrogen is 3.

“Pharmaceutically acceptable,” as used herein, refers to a component that is, within the scope of sound medical judgment, suitable for use in contact with the tissues of humans and other mammals, optionally without undue toxicity, irritation, or immunogenicity and are commensurate with a reasonable benefit/risk ratio.

“Pharmaceutically acceptable salt” means any non-toxic salt that, upon administration to a recipient, is capable of providing, either directly or indirectly, an analog of the instant invention.

“Unsubstituted “Tariquidar” means tariquidar where each atom has a natural isotopic abundance.

Through insight in the mind of the inventor, it has now been discovered that achieving therapeutic efficacy with tariquidar can now be accomplished by producing and administering deuterated analogs of tariquidar. The inventor now believes that such analogs can result in elevated levels in the systemic circulation and greatly inhibit pre-systemic clearance (also known as first pass metabolism) in the gastro-intestinal (GI) tract and the liver and pre and post-systemic biotransformation (or elimination). Such biotransformation of unmodified tariquidar, according to insight by the inventor, results in inactive metabolites through specific enzymatic modification, e.g. by cytochrome monogenases and oxidoreductases in the GI tract and liver. Accordingly, certain deuterated tariquidar analogs have been designed to block metabolic degradation and substantially increase systemic and ONS levels following oral or intravenous or other routes of administration and ultimately result in higher levels in pump-protected compartments. Additionally, according to the inventor, instant analogs will allow effective levels of tariquidar without reaching toxic levels which have more typically been observed, It has also been discovered, according to the mind of the inventor, that certain tariquidar metabolites can be responsible for toxicity. Accordingly, the tariquidar analogs of the present invention should be safer and demonstrate fewer toxicities.

### Tariquidar Analog Nomenclature

For convenience of the reader, tariquidar and tariquidar analogs are named in reference to the structure shown in FIG. 1. Each potential deuteration site is assigned a locant number. Each ring is assigned a letter. There are three linkers, namely an ethyl linker between ring B and ring C (linker 1), a carboximide linker between ring C and ring D (linker 2), and a carboximide linker between ring D and ring F (linker 3). For additional clarity, constituents of a ring which are not part of a linker are deemed to be part of the ring.

### Specific Embodiments

By example, the invention contemplates each of the exemplary embodiments (“EE”) listed below. Each of the EEs is an instant analog according to the invention. It should be understand that where a locant is not specified, it can be a hydrogen or a deuterium.

EE 1. An analog comprising tariquidar having one or more deuterium atoms substituted (i.e. one or more hydrogen atoms are substituted with respective deuterium atoms). Such substitutions can be provided, wherein the normal valence of each atom is maintained.

EE2. The analog of EE1 wherein 1 or 2 deuterium atoms are substituted at locant 4

EE 3. The analog of any of the proceeding EEs wherein 1, 2, or 3 deuterium atoms are substituted at locant 23.

EE4. The analog of any of the proceeding EEs wherein 1, 2, or 3 deuterium atoms are substituted at locant 21.

EE5. The analog of any of the proceeding EEs wherein 1 or 2 deuterium atoms are substituted at locant 11.

EE6. The analog of any of the proceeding EEs wherein 1 or 2 deuterium atoms are substituted at locant 12.

EE7. The analog of any of the proceeding EEs wherein 1 deuterium atom is substituted at locant 6.

EE8. The analog of any of the proceeding EEs wherein 1 deuterium atom is substituted at locant 9.

EE9. The analog of any of the proceeding EEs wherein 1 or 2 deuterium atoms are substituted at locant 4.

EE10. The analog of any of the proceeding EEs wherein 1 or 2 deuterium atoms are substituted at locant 2.

EE11. The analog of any of the proceeding EEs wherein 1 deuterium atom is substituted at one or more of locants 14, 15, 17, or 18.

EE12. The analog of EE11 wherein 1 deuterium atom is substituted at locant 15.

EE13. The analog of EE11 or EE12 wherein 1 deuterium atom is substituted at locant 14.

EE14. The analog of any one of EE11-EE13 wherein 1 deuterium atom is substituted at locant 17.

EE15. The analog of any one of EE11-EE14 wherein 1 deuterium atom is substituted at locant 18.

EE16. The analog of any of the proceeding EEs wherein 1, 2, or 3 deuterium atoms are substituted at locant 36.

EE16. The analog of any of the proceeding EEs wherein 1, 2, or 3 deuterium atoms are substituted at locant 34.

EE17. The analog of any of the proceeding EEs wherein 1 deuterium atom is substituted at locant 27.

EE17. The analog of any of the proceeding EEs wherein 1 deuterium atom is substituted at locant 30.

EE18. The analog of any of the proceeding EEs wherein 1, 2, 3, 4, 5, or 6 deuterium atoms are substituted in ring E plus ring F.

EE19. The analog of any of the proceeding EEs wherein 1 deuterium atom is substituted at locant 40.

EE20. The analog of any of the proceeding EEs wherein 1 deuterium atom is substituted at locant 45.

EE21. The analog of any of the proceeding EEs wherein 1 deuterium atom is substituted at locant 46.

EE22. The analog of any of the proceeding EEs wherein 1 deuterium atom is substituted at locant 47.

EE23. The analog of any of the proceeding EEs wherein 1 deuterium atom is substituted at locant 48.

EE24. The analog of any of the proceeding EEs wherein 1 deuterium atom is substituted at locant 44.

EE25. The analog of any of the proceeding EEs wherein 1 deuterium atom is substituted at locant 27.

EE25. The analog of any of the proceeding EEs wherein 1 deuterium atom is substituted at locant 30.

EE26. The analog of any of the proceeding EEs wherein 1 or 2 deuterium atoms are substituted in ring A.

EE27. The analog of any of the proceeding EEs wherein 1, 2, 3, 4, 5, or 6 deuterium atoms are substituted in ring B.

EE28. The analog of any of the proceeding EEs wherein 1, 2, 3, or 4 deuterium atoms are substituted in ring C.

EE29. The analog of any of the proceeding EEs wherein 1 or 2 deuterium atoms are substituted in ring D.

EE30. The analog of any of the proceeding EEs wherein 1, 2, 3, or 4 deuterium atoms are substituted in ring E.

EE31. The analog of any of the proceeding EEs wherein 1 or 2 deuterium atoms are substituted in ring F.

EE32. The analog of any of the proceeding EEs wherein 1, 2, 3, or 4 deuterium atoms are substituted in Linker 1.

EE 33 The analog of EE1 comprising at least one of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 33 The analog of EE1 comprising at least two of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 34 The analog of EE1 comprising at least three of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 35 The analog of EE1 comprising at least four of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 36 The analog of EE1 comprising at least five of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 37 The analog of EE1 comprising at least six of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 38 The analog of EE1 comprising at least seven of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 39 The analog of EE1 comprising at least eight of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 40 The analog of EE1 comprising at least nine of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 41 The analog of EE1 comprising at least ten of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 42 The analog of EE1 comprising at least eleven of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE 43 The analog of EE1 comprising each of:
i. one or two deuterium atom substituted at locant 4;
ii. one or two deuterium atom substituted at locant 11 or 12 or both 11 and 12
iii. one deuterium atom substituted at locant 18 or 14 or both 18 and 14
iv. one deuterium atom substituted at locant 17 or 15 or both 17 and 15
v. one or two or three deuterium atom substituted at locant 21 or 23 or both 21 and 23
vi. one or two or three deuterium atom substituted at locant 36 or 34 or both 36 and 34
vii. one deuterium atom substituted at locant 6 or 9 or both 6 and 9.
viii. one or two deuterium atom substituted at locant 2 or 3 or both 2 and 3
ix. one deuterium atom substituted at locant 27 or 30 or both 27 and 30.
x. one deuterium atom substituted at locant 40 or 44 or both 40 and 44.
xi. one deuterium atom substituted at locant 45 or 48 or both 45 and 48.
xii. one deuterium atom substituted at locant 46 or 47 or both 46 and 47.

EE44. A composition comprising one or more of any of the analogs of the previous EEs.

EE45. The composition of EE44 wherein the analogs are present in a isotopic purity of greater than 50%.

EE46. The composition of EE44 wherein the analogs are present in a isotopic purity of greater than 70%.

EE47. The composition of EE44 wherein the analogs are present in a isotopic purity of greater than 70%.

EE48. The composition of EE44 wherein the analogs are present in a isotopic purity of greater than 90%.

EE50 The composition of any one of EE44-EE48 wherein there is at least a 20% increase in one or more of in vivo plasma half life in humans, in vitro half life, AUC 0-_{∞}”, and Cmax when compared to the same composition except that the one or more analogs are only unsubstituted tariquidar.

EE51 The composition of any one of EE44-EE48 wherein there is at least a 40% increase in one or more of in vivo plasma half life in humans, in vitro half life, AUC 0-_{∞}”, and Cmax when compared to the same composition except that the one or more analogs are only unsubstituted tariquidar.

EE52 The composition of any one of EE44-EE48 wherein there is at least a 60% increase in one or more of in vivo plasma half life in humans, in vitro half life, AUC 0-_{∞}”, and Cmax when compared to the same composition except that the one or more analogs are only unsubstituted tariquidar.

EE53 The composition of any one of EE44-EE48 wherein there is at least a 100% increase in one or more of in vivo plasma half life in humans, in vitro half life, AUC 0-_{∞}”, and Cmax when compared to the same composition except that the one or more analogs are only unsubstituted tariquidar.

EE54. The composition of any one of EE44-EE53 further comprising a therapeutic agent.

Deuteration Designation and Enrichment and Chemical Nomenclature 0097 Unless otherwise stated, when a position is designated specifically as “D” or “deuterium”, the position is understood to have deuterium at an abundance that is at least 3000 times greater than the natural abundance of deuterium, which is 0.015% (i.e., at least 45% incorporation of deuterium).

Any atom not designated as deuterium should be understood to be present at its natural isotopic abundance.

Instant analogs taught herein will inherently contain small amounts of isotopologues (e.g. having isotopes present at their natural isotopic abundance at locants other than those taught as substituted herein).

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the analogs of this invention, any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

Compositions of the invention can optionally be a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above, the relative amount of such isotopologues in toto will be less than 55% of the compound. In other embodiments, the relative amount of such isotopologues in toto will be less than 50%, less than 47.5%, less than 40%, less than 32.5%, less than 25%, less than 17.5%, less than 10%, less than 5%, less than 3%, less than 1%, or less than 0.5% of the compound.

Unless otherwise indicated, when a disclosed compound is named or depicted by a structure without specifying the stereochemistry and has one or more chiral centers, it is understood to represent all possible stereoisomers of the compound.

The analogs of the present invention may contain an asymmetric carbon atom, for example, as the result of deuterium substitution or otherwise. As such, analogs of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final analogs or to starting material or intermediates.

### Tariquidar Synthesis.

Tariquidar can be synthesized as described in 6,218,393. The skilled artisan will now readily recognize that the deuterated analogs of the present invention can be made by starting with starting compounds deuterated at the appropriate position.

Another useful method for synthesis of the instant analogs is to generally follow the methods of Puentes et al. (Bioorganic & Medicinal Chemistry Letters 21 (2011) 3654-3657).

Another useful method for synthesis of the instant analogs is to generally follow the methods of Bauer et al. (Bioorg Med Chem. 2010 August 1; 18(15): 5489-5497).

Another useful method of synthesis is described by Bernd et al. (“Synthesis and Small-Animal Positron Emission Tomography Evaluation of [11C]-Tariquidar as a Radiotracer to Assess the Distribution of P-Glycoprotein at the Blood-Brain Barrier.” Journal of medicinal chemistry 52.19 (2009): 6073-6082. PMC. Web. 6 Mar. 2018).

Another useful method for synthesis of the instant analogs is to generally follow the methods shown in FIG. 4. The reactants (e.g. Compounds 1, 2, 5, and 6) shown in FIG. 3 have locants numbered according to those in the final tariquidar analog as shown in FIG. 1. This method is described in further detail by way of example in Example 1. Using this method, any of the tariquidar analogs taught herein can be synthesized by selection of Compounds 1, 2, 5, and 6 which are deuterated appropriately. To aid in the selection of the appropriately deuterated reactants, the atoms in the reactant are numbered according to the locant numbers in tariquidar (showing their location in the finished synthetic product).

Deuterium can also be incorporated to various positions, selectively or non-selectively through a proton-deuterium exchange method known in the art.

### Pharmaceutical Salts

Analogs and compositions of the present invention can be prepared as pharmaceutically acceptable salts.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The pharmaceutically acceptable salt may also be a salt of a compound of the present invention and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methylamine, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Compositions, Dosage Forms and Carriers

The invention also provides pharmaceutical compositions comprising an effective amount of a compound (e.g. instant analog) of the invention and a pharmaceutically acceptable carrier. The carrier(s) are also acceptable in the sense of being compatible with the other ingredients of the formulation.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. If required, the solubility and bioavailability of the analogs of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Nauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Optionally, instant compositions are in a solid or liquid form. For example, solid forms include tablets or particle-containing capsules formulated for oral administration. As another example, liquid forms include suspensions or emulsions, e.g. comprising emulsifiers or surfactants such as polysorbates or hydroxypropylmethylcellulose (Sane R, Agarwal S, Elmquist W F. Brain Distribution and Bioavailability of Tariquidar after Different Routes of Administration in the Mouse. Drug Metabolism and Disposition. 2012; 40(8):1612-1619. doi:10.1124/dmd.112.045930).

Optionally, an instant composition comprises about 10 mg to about 20000 mg of a tariquidar analog, e.g., about 25 mg to about 1000 mg. Other contemplated compositions are those that provide dosing as taught herein.

Optionally, an instant composition is provided in a container. For example, the container can be a sealed container, a syringe (e.g. configured for IV administration), an IV bag (e.g. mixed with a chemotherapeutic agent), a pharmacy vial configured for pills (e.g. having a child-proof lid), or a pharmacy vial configured for liquid formulation (e.g. having a sealed lid configured for puncture by a syringe).

Optionally, an instant composition is a liquid composition provided in a container other than an assay type container (e.g. other than an NMR tube or cuvette).

In one embodiment, an instant composition comprises a tariquidar analog and a second therapeutic, e.g. any second therapeutic taught herein for co-administration with the tariquidar analog.

In one embodiment, an instant composition is configured for any route of administration taught herein.

### Routes of Administration

The skilled artisan, with the teaching herein, will readily recognize the routes of administration of analogs and compositions of the present invention.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the tariquidar analogis administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, may also be added. For oral administration in a capsule form, useful diluents include lactose and dried cornstarch. When aqueous suspensions are administered orally, the active ingredient is optionally combined with emulsifying and suspending agents. If desired, certain sweetening and/or flavoring and/or coloring agents may be added.

Compositions suitable for oral administration include lozenges comprising the ingredients in a flavored base, usually sucrose and acacia or tragacanth; and pastilles comprising the active ingredient in an inert base such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the analogs of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of an instant tariquidar analog may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

### Dosing

A tariquidar analog can be provided in a specific dose. The dose amount depends, among other things, upon the route of administration. Using routine methodology, the skilled artisan can readily determine dosing amounts based on resultant plasma levels. With this said, dosing should a target plasma Cₘₐₓ of greater than 10 ng/ml or greater than 20 ng/ml or greater than 40 ng/ml. With the instant invention, these plasma levels can now be achieved by oral administration of less than 2 gm or less than 1 gm or less than 0.5 gms of a tariquidar analog.

Co-Administration 00134 With the teaching herein, the skilled artisan will readily recognize the value of combining instant analogs and compositions with a second therapeutic agent or agents.

Pumps of the P-gp and/or BCRP type are known to diminish accumulation of (or therapeutic exposure of) certain therapeutic agents (e.g. those that are pump substrates) in pump-protected target tissues, while exceeding tolerable exposure in non-pump-protected, non-target tissues. As non-limiting examples of such targets are brain and solid tumors. Accordingly, analogs and compositions of the present invention are useful when co-administered with one or more of such therapeutic agents.

Examples of therapeutic agents that are useful in combination with instant analogs or compositions are tyrosine kinase inhibitors such as dasatinib, gefitinib, imatinib, pazopanib, sorafenib, sunitinib, erlotinib, and vandetanib, for example.

Other examples of therapeutic agents that are useful in combination with instant analogs or compositions are other anti-neoplastic agents such as crizotinib, docetaxel, doxorubicin, imidazotetrazine, ispinesib, paclitaxel, tazemetostat, temozolomide, and topotecan, for example.

Optionally, the second therapeutic agent is an antitumor drug. Examples of antitumor drugs include vinca alkaloids, anthracyclines, taxol and derivatives thereof, podophyllotoxins, mitoxantrone, actinomycin, colchicine, gramicidine D, amsacrine or any drug having cross resistance with above drugs characterized by the so-called multidrug resistance (‘MDR’) phenotype.

Other examples of therapeutic agents that are useful in combination with instant analogs or compositions are antiviral and anti-retroviral drugs such as abacavir, amprenavir, lamivudine, ritonavir, and zidovudine, for example.

Other examples of therapeutic agents that are useful in combination with instant analogs or compositions are opioid receptor agonists such as loperamide, morphine, and n-desmethylloperamide, for example.

In another embodiment, the instant invention provides separate dosage forms of a tariquidar analog of this invention and one or more of any of the above-described second therapeutic agents or classes of therapeutic agents, wherein the tariquidar analog and second therapeutic agent are associated with one another. The term “associated with one another” as used herein includes separate dosage forms are packaged together or otherwise attached to one another such that it is readily apparent that the separate dosage forms are intended to be sold and administered together (within less than 24 hours of one another, consecutively or simultaneously).

According to the present invention, one or more therapeutic agents and a tariquidar analog can be co-administered with a second or third or fourth drug such that all drugs are present in the subject at the same time. For example, a tariquidar analog can be administered before, after, or at the same time as the second drug.

### Methods of Use

In one embodiment, the present invention provides a method comprising administering an instant tariquidar analog to a subject in need thereof.

In another one embodiment, the present invention provides a method of enhancing the efficacy of a therapeutic agent comprising co-administering an instant tariquidar analog and a therapeutic agent, where optionally, the therapeutic agent is a substrate for a P-gp pump or a BCRP pump.

For example, an instant tariquidar analog can be used for sensitizing multidrug-resistant cancer cells to chemotherapeutic agents.

Optionally, the subject is a mammal or a human.

Optionally, the administration is oral or injection.

Optionally, the instant tariquidar analog is administered in the form of a pharmaceutically acceptable composition taught herein.

Optionally, the method comprises co-administering a second therapeutic, e.g., a second therapeutic taught herein.

### Superior and Unexpected Properties

There has been a long recognized, unmet need to provide a means of increasing drug accumulation in or distribution to certain pump-protected target tissues like solid tumors, sites in the central nervous system and stem cells. The mechanisms which limit such accumulation or distribution are well known, i.e. there exist certain efflux pumps which naturally occur or are upregulated (e.g. especially in certain pathologies or induced by drug exposure) that pump such drugs away from the desired target. It has been discovered, according to the mind of the inventor, that instant analogs and compositions now provide remarkable superior properties over previously known pump inhibitors or previously used tariquidar compounds and compositions. It is now possible to use the instant analogs and compositions in a manner that increases therapeutic agent accumulation in or distribution to the pump-protected target regions.

Due to the aforementioned unpredictability of results obtained from deuteration of active pharmaceutical ingredients, it is quite surprising that tariquidar analogs of the present invention can improve the systemic exposure and pharmacodynamic profile relative to unsubstituted tariquidar. The improved pharmacokinetics of deuterated analogs of tariquidar can be demonstrated by any of improved in vivo half life, increased AUC and Cmax, reduced first pass metabolism in the GI tract and liver, and reduced systemic metabolism demonstrated in vivo or in vitro (e.g. in a microsomal or supersomal assay). Deuterated analogs of tariquidar can improve the pharmacodynamics of a coadministered therapeutic agent by increasing exposure of the therapeutic agent in pump-protected tissues and improving therapeutic response when compared to non-deuterated tariquidar.

Accordingly, it is now possible to achieve and maintain therapeutic levels of a therapeutic agent in pump-protected tissues and, at the same time, maintaining or reduce levels of the therapeutic agent in the systemic circulation and non-pump-protected tissues, which results in a decrease in toxicity and other adverse events. This is due to the ability to redistribute the therapeutic agent from peripheral to pump-protected target tissues while administering lower doses of the therapeutic agent (that would otherwise be too toxic to be useful). To state this differently, the therapeutic window of certain therapeutic agents can be widened, where it previously was marginal or non-existent. In order to obtain therapeutic levels in the target tissues with native (unsubstituted) tariquidar, it would be necessary to administer high doses of the therapeutic agent resulting in toxic drug levels in plasma and peripheral, non-target tissues. With the superior properties of the instant tariquidar analogs, the therapeutic index is greatly enhanced.

In some embodiments, instant analogs can result in an increase in serum half life of 20% or more than 40% (when compared to a similar dose, administration, and composition of the unsubstituted tariquidar). Similarly, the AUC of instant analogs can be increased by 20% or more than 40%. Similarly, when instant analogs and compositions are co-administered with a therapeutic drug which is a substrate for P-gp or BCRP-like pumps, the levels of such a therapeutic drug in pump-protected tissues such as the brain or in a solid tumor can be increased as much as 20% or 40% or more.

By way of example, increase in brain levels of the following compounds can be remarkably increased when co-administered with instant analogs and compositions (when compared to co-administration with a similar dose, administration, and composition of the unsubstituted tariquidar): dasatinib, gefitinib, imatinib, pazopanib, sorafenib, sunitinib, and vandetanib, crizotinib, docetaxel, doxorubicin, imidazotetrazine, ispinesib, paclitaxel, tazemetostat, temozolomide, and topotecan, abacavir, amprenavir, lamivudine, ritonavir, zidovudine, loperamide, morphine, and n-desmethylloperamide.

As discussed above, it has also been discovered, according to the mind of the inventor, that certain tariquidar metabolites are responsible for toxic effects of tariquidar administration and that the instant analogs should be safer and exhibit less toxicities.

### EXAMPLES

The following examples are intended to illustrate but not to limit the invention. Moreover, scientific discussions below of underlying mechanisms gleaned from the data are also not meant as limitations of the inventions described here.

### Example 1. Synthesis of Instant Analogs and Compositions

This example demonstrates a synthetic method for making tariquidar analogs, deuterium substitutions based upon the deuteration of the starting compounds. The synthesis is shown in FIG. 3 and FIG. 4. The numbered compounds can be named as follows:
Compound 1 6,7-Dimethoxy-1,2,3,4-tetrahydro-isoquinoline
Compound 2 1-(2-Bromo-ethyl)-4-nitro-benzene
Compound 3 6,7-Dimethoxy-2-[2-(4-nitro-phenyl)-ethyl]-1,2,3,4-tetrahydro-isoquinoline
Compound 4 6,7-Dimethoxy-2-[2-(4-nitro-phenyl)-ethyl]-1,2,3,4-tetrahydro-isoquinoline
Compound 5 4,5-dimethoxy-2-nitrobenzoic acid
Compound 6 N-{4-[2-(6,7-Dimethoxy-3,4-dihydro-1H-isoquinolin-2-yl)-ethyl]-phenyl}-4,5-dimethoxy-2-nitro-benzamide
Compound 7 2-Amino-N-{4-[2-(6,7-Dimethoxy-3,4-dihydro-1H-isoquinolin-2-yl)-ethyl]-phenyl}-4,5-dimethoxybenzamide
Compound 8 3-quinolinecarbonyl chloride
Compound 9 N-[2-[[4-[2-(6,7-dimethoxy-3,4-dihydro-1H-isoquinolin-2-yl)ethyl]phenyl]carbamoyl]-4,5-dimethoxyphenyl]quinoline-3-carboxamide

Step A:

A 12 L three-neck flask is charged with compound 2(475 g, 2.065 mol), compound 1 (474.8 g, 2.065 mol), K2CO3 (314 g, 2.273 mol), KI (68.6 g, 0.413 moL) and DMF (2.5 L) and the resulting mixture is heated to 70° C. and stirred for 2.5 hours. After LC-MS showed that the reaction was complete, the mixture is cooled to 50° C. and methanol (620 mL) is added. Then the mixture was cooled to 30° C. and water (4.75 L) was added. The resulting suspension was cooled to 10° C. and for 1 hour. The solid is filtered, washed with water (2×2.5 L) and air dried for 2 days to afford the compound 3 (630 g, 89.1%) as a yellow solid.

Step B

To a solution of compound 3 (630 g, 1.84 mol) in THF/ethanol (8 L at 1:1) is added Pd/C (10%, 50% wet, 30 g). The mixture is stirred under an atmosphere of hydrogen (1 atm, balloon) at 15-20° C. for 4 h. The reaction mixture is filtered through a pad of Celite and the pad was washed with tetrahydrofuran (1.0 L). The filtrate was concentrated to 3 volumes under vacuum and hexanes (4.0 L) was added. The resulting slurry is cooled to 0° C. and stirred for 1 h. The solid is filtered and washed with hexanes (2×500 mL) and air dried overnight to afford the compound 4 (522 g, 90.8%) as an off-white solid.

Step C.

To a solution of compound 4 is added compound 5 in thionyl chloride, tetrahydrofuran, triethylamine and allowed to react to form Compound 6. The resultant slurry is cooled to 0° C. and stirred for 1 hr. The solid is filtered and washed with hexanes (2×500 mL) and air dried overnight.

Step D

A solution of compound 6 is reacted in the presence of H2, palladium on carbon catalyst, and ethyl acetate/methanol and allowed to react to form Compound 7

Step E

To a solution of compound 7 is added Compound 8 in the presence of tetrahydrofuran, triethylamine; ammonia, and ethanol at 50° C. and allowed to react to form compound 9 (tariquidar). The resultant slurry is cooled to 0° C. and stirred for 1 h. The solid is filtered and washed with hexanes (2×500 mL) and air dried overnight.

### Example 2. Demonstration of Superior Properties of Instant Analogs and Compositions: In Vivo ADMET

Pharmacologic studies are performed in methods which are a modification of those taught by Ward K W et al (2001 Xenobiotica 31:783-797) and Ward and Azzarano (JPET 310:703-709, 2004). Briefly, instant analogs are administered solutions in 10% aqueous polyethylene glycol-300 (PEG-300) or 6% Cavitron with 1% dimethyl sulfoxide, or as well triturated suspensions in 0.5% aqueous HPMC containing 1% Tween 80. Blood samples are collected at various times up to 48 h after drug administration; plasma samples are prepared and at “70° C. until analysis.

Mice. Instant analogs are administered to four groups of animals by oral gavage (10 ml/kg dose volume). Three groups receive instant analogs as a suspension at 3, 30, or 300 mg/kg, and the fourth group receive instant analogs as a solution in Cavitron at 3 mg/kg. Blood sampling in mice is performed via a tail vein at 0.5, 1, 2, 4, 8, 24, and 32 h postdose.

Dogs. Dogs receive instant analogs by lavage (4 ml/kg) on three separate occasions with dosages at 3 and 30 mg/kg as a suspension and 3 mg/kg as a solution in Cavitron. Blood samples are obtained from a cephalic vein and from the hepatic portal vein catheter before dosing and at 5, 15, 30, and 45 min and 1, 1.5, 2, 3, 4, 6, 8, 10, 24, 32, and 48 h postdose.

Rats. A total of seven groups of animals receive instant analogs by oral gavage (10 ml/kg). Three groups receive instant analogs as a suspension at 3, 30, or 300 mg/kg, and a fourth and fifth group each receive instant analogs as a solution in Cavitron or PEG-300, respectively, at 3 mg/kg. A sixth and seventh group of rats with indwelling hepatic portal vein catheters receive instant analogs by oral gavage (10 ml/kg) as a suspension at 3 or 30 mg/kg, respectively. Blood sampling in rats are performed via a lateral tail vein; samples are also obtained from the hepatic portal vein catheter. Blood samples are obtained before dosing and at 5, 15, 30, and 45 min, and 1, 1.5, 2, 3, 4, 6, 8, 10, 24, and 32 h Dogs receive instant analogs by lavage (4 ml/kg) on three separate occasions with dosages at 3 and 30 mg/kg as a suspension and 3 mg/kg as a solution in Cavitron. Blood samples are obtained from a cephalic vein and from the hepatic portal vein catheter before dosing and at 5, 15, 30, and 45 min and 1, 1.5, 2, 3, 4, 6, 8, 10, 24, 32, and 48 h postdose.

Monkeys. Monkeys receive instant analogs by oral gavage (8 ml/kg dose volume) on three separate occasions at dosages of 3 and 30 mg/kg as a suspension and 3 mg/kg as a solution in Cavitron. Blood samples are obtained from a femoral vein via an indwelling catheter and from the hepatic portal vascular access port before dosing and at 5, 15, and 30 min and 1, 1.5, 2, 4, 6, 8, 10, 24, 32, and 48 h postdose.

Humans. Healthy volunteers receive instant analogs orally at doses ranging from 25 mg to 1000 mg. Blood samples are obtained and analyzed for analog concentrations at 0, 15 min, 30 min, 45 min, 60 min, 90 min, 120 min, 180 min, 2 hr, 4 hr, 6 hr, 8 hr, 12 hr, 24 hr, and 48 h after administration.

Analytical Methods

Instant analogs are isolated from samples by precipitation with acetonitrile and quantified by LC/MS/MS coupled with an atmospheric pressure chemical ionization interface (475° C.). Internal standards [in acetonitrile/10 mM ammonium formate, pH 3.0; 95:5 (v/v)] are added to 50 μl samples and vortexed and centrifuged for 30 min at 4000 rpm. The supernatants are injected onto the LC/MS/MS system using an HTS PAL autosampler (CTC Analytics, Zwingen, Switzerland) coupled to an Aria TX2 high-throughput liquid chromatographic system using turbulent flow technology (Cohesive Technologies, Franklin, Mass.) in focus mode. The mobile phase consists of a mixture of 0.1% formic acid in water and 0.1% formic acid in acetonitrile. The turbulent flow column is a 0.5×50-mm Cyclone P column (Cohesive Technologies) in series to a 2×20 mm, 4 μm Polar RP (Phenomenex, Torrance, Calif.) analytical column. Positive-ion multiple reaction monitoring is used for the detection of instant analogs and internal standard and the selected precursor and product ions are m/z 564 and 252, respectively. Using a (1/x) weighted linear regression analysis of the calibration curve, linear responses in analyte/internal standard peak area ratios are observed for instant analog concentrations ranging from 2 to 10,000 ng/ml.

Alternatively, useful analytical methods to demonstrate the surprising and superior properties of the instant tariquidar analogs are modifications of the methods as described by Stokvis et al, J Mass Spectr 2004: 39: 1122-1130.

Alternatively, useful analytical methods to demonstrate the surprising and superior properties of the instant tariquidar analogs are modifications of the methods using solid phase extraction followed by liquid chromatography with tandem mass-spectrometric detection as described by Stewart et al. Clinical Cancer Research 6:4186-4191 (2000).

Pharmacokinetic Data Analysis.

Concentration versus time profiles are obtained for each analyte in each animal and noncompartmental analysis is performed using WinNonlin Professional version 3.3 (Pharsight, Mountain View, Calif.) to recover area under the curve (AUC), Cmax and other parameters. Dose-normalized AUC (DNAUC) (minutes×kilograms per liter) is determined by dividing AUC by dose (milligrams per kilogram) and multiplying by 1000. For studies for which both portal and systemic data are available, absorption and first-pass hepatic extraction is estimated as described by (Ward et al., 2001 (Dug Metab Dispos 29:82-88.).

Results

There is substantial species variability between the mouse, rat, dog, monkey, and human species. Nevertheless, according to insight in the mind of the inventor, instant analogs have one or more of the following superior properties when compared to the same dose of (unsubstituted) tariquidar: (1) increased AUC; (2) increased DNAUC; (3) decreased pre-systemic clearance; (4) increased Cmax; (5) increased Tmax; (6) increased half-life and (7) increased absorption rate.

### Example 3 Demonstration of Superior Properties of Instant Analogs and Compositions: In Vitro Metabolism

This study is designed to predict biotransformation of tariquidar in a model for hepatic metabolism and to compare it with the instant analogs. Additionally, the effect of CYP450 inhibitors such as ritonavir on the metabolic conversion is examined.

In one study, a human liver microsomal system is used; the microsomes are obtained from a commercial source (Thermo Scientific). Additionally, liver microsomes from the wildtype, Cyp3a knockout and Cyp3a KO; and CYP3A4 transgenic mice are prepared.

Bioconversion of tariquidar and instant analogs at various concentrations are incubated with microsomes in a NADPH regenerating system as described by Cheng et al. (Nat Protoc 2009; 4: 1258-1261) and Hendrikx et al (Int J Cancer 2013; 132: 2439-2447).

Tariquidar and instant analogs are monitored using liquid chromatography coupled Ultraviolet-photodiode array (LC-UV-PDA), fluorescence detection (FD) and LC-mass spectrometry or as described in Example 2.

The in vitro metabolic studies are performed in the absence and in the presence of inhibitors such as CYP3A4: ritonavir, ketoconazole; CYP3A4/CYP2C19: fluconazole; CYP2C19/CYP2A6: fluoxetine; CYP2C8: clopidogrel to identify indirectly the enzymes responsible for bioconversion.

Results.

The following results, through insight in the mind of the inventor, show the following: Instant analogs have one or more of the following superior properties when compared to tariquidar (1) one or more minor metabolites appear upon incubation of instant tariquidar analogs, but at a greatly reduced rate and abundance; and (2) certain metabolites that are present upon incubation of the tariquidar samples are absent or nearly absent from the instant tariquidar analog samples. The results when tariquidar analogs are incubated in the presence of various CYP inhibitors suggest that the biotransformation is the result of an enzymatic process.

### Example 4 Demonstration of Superior Properties of Instant Analogs and Compositions: Facilitating Accumulation/Distribution of a Co-Administered Therapeutic Agent to Pump-Protected Target Sites

This example uses co-administration of instant analogs (in comparison to unsubstituted elacridar) with R-11C-verapamil, a substrate of P-gp. It is well known that the presence of P-gp at the blood brain barrier greatly limits the R-11C-verapamil bioavailability in the brain. Instant tariquidar analogs and unsubstituted tariquidar are individually administered with an R-11C-verapamil in i.v. microdose to wild type rats. The rats are either pretreated with either (1) instant analogs or (2) unsubstituted tariquidar at various doses (e.g. 5 or 10 mg/kg). The plasma and brain concentrations of R-11C-verapamil are measured at various times (e.g. at 30 min and 1, 2, 4, 8, and 12 h after dose). The methodologies used here are modifications of the methods used by Bankstahl et al, in Journal of Nuclear Medicine: Official Publication, Society of Nuclear Medicine [16 Jul. 2008, 49(8):1328-1335].

By insight in the mind of the inventor, instant analogs provide remarkable increase in brain concentration of verapamil when compared to similar administrations of unsubstituted tariquidar.

We claim:

1. An analog with the structure of represented by formula 1: 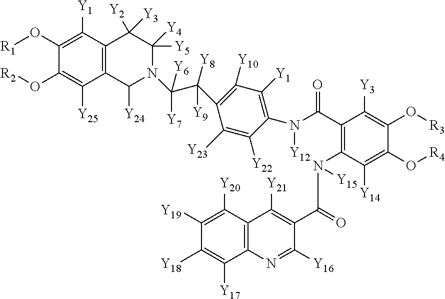
or a pharmaceutically acceptable salt thereof, comprising at least one deuterium atom wherein
each Y is independently selected from hydrogen or deuterium; and
each R is independently selected from CH₃, CH₂D₁, CH₁D₂, and CD₃.

2. The analog of claim 1 wherein Y6-Y9 are each deuterium.

3. The analog of claim 1 wherein R1-R4 are each CD3.

4. The analog of claim 1 wherein Y1-Y25 are each CD3

5. The analog of claim 1 wherein R1-R4 are each CD3 and Y1-Y25 are each CD3.

6. The analog of claim 1 with the structure of any one of EE 1-EE 43.

7. The analog of claim 6 wherein the analog has at least a 20% increase in AUC 0-^{∞} when compared to unsubstituted tariquidar when the composition is administered orally in at least one of a human, a rat, or a mouse and at a dosage level of at least one of 1 mg/kg, 5 mg/kg, or 10 mg/kg.

8. The analog of claim 7 wherein the increase in AUC 0-^{∞} is at least 40%.

9. The analog of claim 6 wherein the analog has at least a 20% increase plasma half life when compared to unsubstituted tariquidar when administered orally in at least one of a human, a rat, or a mouse and at a dosage level of at least one of 1 mg/kg, 5 mg/kg, or 10 mg/kg.

10. The analog of claim 6 wherein the increase in plasma half life is at least 40%.

11. The analog of claim 6 wherein when administered orally in at least one of a human, a rat, or a mouse at a dosage level of at least one of 1 mg/kg, 5 mg/kg, or 10 mg/kg, and coadministered orally with erlotinib at 20 mg/kg, said administration results in an increase in Kp, brain of erlotinib of at least 20%.

12. The analog of claim 6 wherein when administered orally in at least one of a human, a rat, or a mouse at a dosage level of at least one of 1 mg/kg, 5 mg/kg, or 10 mg/kg, and coadministered intravenously with verapomil at 1 mg/kg, said administration results in an increase in Kp, brain of verapomil of at least 20%.

13. The analog of claim 11 wherein the increase in Kp, brain is at least 40%.

14. The analog of claim 12 wherein the increase in Kp, brain is at least 40%.

15. The analog of claim 6 further comprising one or more therapeutic agents.

16. The analog of claim 6 having an isotopic purity of greater than 80%.

17. A method of treating a patient in need comprising administering an analog of claim 6 and co-administering a therapeutic amount of a therapeutic agent.

18. The method of claim 17 wherein the therapeutic agent is one or more of a tyrosine kinase inhibitor, an anti-neoplastic agent, an anti-tumor agent, an antiviral agent, and an anti-retroviral drug,

19. The method of claim 17 wherein the therapeutic agent is one or more of Paclitaxel, topotecan, dasatinib, gefitinib, imatinib, pazopanib, sorafenib, sunitinib, vandetanib, erlotinib, crizotinib, docetaxel, doxorubicin, imidazotetrazine, ispinesib, paclitaxel, tazemetostat, temozolomide, topotecan, vinca alkaloid, anthracyclines, taxol, taxol derivatives, podophyllotoxins, mitoxantrone, actinomycin, colchicine, gramicidine D, amsacrine, abacavir, amprenavir, lamivudine, ritonavir, zidovudine, loperamide, morphine, and n-desmethylloperamide.
